(19) United States
(12) Patent Application Publication
(10) Pub. No.: US 2012/0109016 A1
(43) Pub. Date: May 3, 2012

(54) METHOD AND APPARATUS FOR IMPROVING TRUNCAL CONTROL

(76) Inventor: Marilyn Hintz, Scottsdale, AZ (US)

(21) Appl. No.: 12/939,160

(22) Filed: Nov. 3, 2010

(51) Int. Cl. A61B 5/103

(52) U.S. Cl. 600/595

(57) ABSTRACT

An exercise monitoring apparatus for measuring proper abdominal strengthening affords a customer an improved method for developing better abdominal muscles to treat back pain, avoid injuries and improve physical performance. The exercise apparatus has a stretchable belt with first and second ends having fasteners to attach the belt to the athlete's waist, the fasteners accommodating a variety of waist sizes. The belt has a wider middle portion formed by wider surfaces of the fabric, the wider portion forming a pocket with an exit at a small opening. The pocket accommodates an airtight sac containing gas-filled, compressible foam. A manometer is attached to the sac via a tube that passes through the small opening around the sac, and the manometer displays data on pressure changes as the customer uses the belt.

### RELATED APPLICATION

This application claims the benefit of U.S. Provisional Application No. filed Nov. 2, 2009, and incorporates the prior application in its entirety.

### TECHNICAL FIELD

The present invention relates to the field of exercise monitoring, more particularly to an apparatus and method for monitoring pressure generation while strengthening core muscles.

### BACKGROUND

Back pain is one of the most common medical problems, affecting eight of ten people at some point during their lives. About 25% of Americans are affected by back pain each year. They spend more time at the doctor's office for back pain than for any other medical condition except high blood pressure and diabetes (which are directly related to obesity). Unfortunately, the expected improvements in patient health have not been achieved through the current services and procedures, resulting in a dramatically increased use of narcotics, injections and surgery over the past decade. More money is spent treating neck and back pain than almost any other medical condition, and most neck and back pain is preventable. There are more than 15 million outpatient physician visits for back pain alone in a given year in the US. Even though only a fraction of people with back pain are good candidates for surgery, complicated spine operations are on the rise. Complicated surgeries can result in unwanted scarring that gives rise to more pain; repeat surgeries are at 20%.

Back and neck problems are the second leading cause of disability and the leading cause of job-related disability, costing the US more than USD50 billion each year.

About a half a billion dollars a year is spent on the sale of exercise equipment through infomercials and USD 207 million of that is for abdominal machines alone. However, none of these machines provide detailed instructions on how to properly activate “core” muscles. When the deep inner core muscles are not activated properly, strain is placed on the spine and can results in injuries and/or pain.

Currently there is a subjective measure of core muscle activation that is used universally in physical therapy: The clinician places a hand between the lower back and the floor or table while instructing the patient to continue to apply pressure or “squeeze” the back into the clinician's hand and maintain that position while performing various exercise motions. This is labor intensive for the clinician. When the patient exercises unsupervised, she quickly forgets the fine points of such exercise and returns to improper activation, discomfort and inadequate physical performance.

A device used in some physical therapy clinics for treatment of low back pain is a blood pressure cuff, but that has the following limitations: 1) too short a hose which causes the body to twist and cause an inaccurate reading while performing exercises, 2) an extra hose that gets in the way, 3) too small print on the gauge to read the biofeedback data on, in addition to also being in units of mmHg and no indicators of the ideal range to reach throughout exercise, 4) dependence on clinician to place the air filled sac of the blood pressure cuff in the correct part of the body to get a accurate biofeedback, 5) too much error in feedback because the blood pressure cuff requires someone to manually pump air into the sac, and 6) manual pumping producing variable amounts of air and variable results, and 7) mishandling the bulb valve decreasing pressure.

There recently has been introduced a device called the ‘Stabilizer’ that works like a blood pressure cuff that has earlier been utilized as an objective biofeedback when the transverse abdominals are activated during physical therapy sessions (U.S. Pat. No. 5,338,276). This new device apparently depends on the clinician to correctly place the device on the body, the air sac seems too large and causes distortion of body position, and the biofeedback is given in units of mmHg, without a clear guide to desired pressure, although a new model has red lines to indicate a range).

### SUMMARY OF THE INVENTION

In accordance with the present invention, an exemplary apparatus for monitoring pressure generation needed for proper abdominal strengthening is disclosed. The apparatus has a stretchable belt with a first end, second end, a first side, a second side, inside surface, outside surface. The first and second ends have fasteners to attach the belt around the customer's waist, and the fasteners are so arranged as to accommodate a variety of waist sizes. The first side has a small opening; and the belt has a wider portion in the middle formed by wider surfaces of the fabric, the wider portion forming a pocket with an exit at the small opening. The pocket accommodates a finable sac containing gas-filled foam. A manometer attaches to the sac via a tube that passes out through the small opening, the manometer displaying data on pressure changes as the customer uses the belt.

Optionally the center of the wider portion of the apparatus has a marker to align substantially with an athlete's spine. The marker is painted, sewn, silk screened or glued onto the belt. The belt is formed from Neoprene® polychloroprene or other material with similar characteristics.

The two surfaces of the belt comprise two at least attached pieces of Neoprene®. The tube connecting the foam sac and the manometer is sufficiently long for the person wearing the belt to see the manometer while exercising and without twisting. Such a tube is at least 30 inches long. Alternatively, the tube is between 28 and 45 inches long.

The tube passes from the sac through the belt to a side hole for exiting the belt.

The manometer has a face with demarcated directions and pressure units. Alternatively, the small opening for the tube is lengthened to the width of the sac, thereby permitting removal of the sac assembly. The fasteners are Velcro® hooks and loops, hooks and eyes, buttons, snaps, etc.

In another embodiment, the monitoring apparatus has a) a stretchable belt formed from a single piece of Neoprene® with a first end, second end, a first side, a second side, inside surface, outside surface; b) the first and second ends having fasteners to attach the belt to the athlete's waist, the fasteners being so arranged as to accommodate a variety of waist sizes; c) a pocket sized to contain a sac filled with compressible foam, the pocket having an inner surface and an outer surface, the inner surface being attached to the outside belt material of the belt, so that the foam sac is compressed against the back in use, the pocket further having an upper edge; and d) a manometer attached to the sac via a tube that passes out through the open upper edge of the pocket, the manometer representing data on pressure changes as improper activation, initiation of activation, and proper and consistent activation of transverse abdominals, as the customer uses the belt.

The apparatus also has a marker to align substantially with an athlete's spine. This marker is painted, silk screened, sewn or glued onto the belt. The apparatus can have two surfaces of the belt comprise two attached pieces of Neoprene® material. The tube connecting the foam sac and the manometer is sufficiently long for the person wearing the belt to see the face of the manometer while exercising and without twisting or having to torque their neck. The tube is about 28-45 inches long.

The apparatus' fasteners can be Velcro® hooks and loops, Velcro hooks alone on the fabric, hooks and eyes, buttons, or snaps.

In another embodiment, there is provided a method of monitoring to maintain the proper pressure of the abdominal muscles during exercise. The first step is providing an apparatus including a stretchable, two-layered belt with a wide middle portion for encapsulating a foam sac, the foam sac being attached to a tube whose other end attaches to a manometer, either end of the belt having fasteners, and the middle of the belt having a marker for centering the foam air sacover the spine; putting on the belt by pulling the belt around the midsection;
attaching the fasteners for a snug fit;
checking to assure that the center marker is over the spine and optionally rearranging the belt until the belt is so positioned;
positioning the body of the customer to perform an exercise involving the transversal abdominals or the multifidi; Positioning the manometer so that the customer can see the manometer face without turning the face from the midline; beginning the exercise; and
watching the manometer face and adjusting the exercise performance until the manometer face indicates proper and consistent activation of muscles.

### BRIEF DESCRIPTION OF THE DRAWINGS

For a further understanding of the objects and advantages of the present invention, reference should be made to the following detailed description, taken in conjunction with the accompanying drawing, in which like parts are given like reference numbers and wherein:

FIG. 1 is an overview of the monitoring apparatus;

FIG. 2 is a schematic of the monitoring apparatus, showing the Velcro® ends and the central compressible foam sac;

FIG. 3 shows a variation on the design and assembly of the belt.

FIG. 4 shows another variation on the design; and

FIG. 5 shows another variation on the design and assembly of the belt.

### DETAILED DESCRIPTION OF THE PREFERRED EMBODIMENTS

As an athletic trainer and strength coach, I found my athletes and patients were not retaining the proper activation of their abdominals throughout various movements and exercise. Like most therapists I would tell them to push my hand into the ground with their back. I grew to realize that they would not maintain that amount of activation when I removed my hand, so I needed to develop another method of teaching proper abdominal muscle activation.

I remembered my professor in grad school teaching us to use a blood pressure cuff and watch the pressure dial to make sure athletes maintain the contraction throughout “dead bug” exercise or while lowering their shoulders back to the ground following a “mini crunch.” However, I observed several problems with this device. First, the hose was not long enough for the athlete to stay in position and observe the dial. If the athlete were to see the dial, he would have to twist and get inaccurate readings and less benefit. Second, the blood pressure has two hoses and an air bulb which confuses the customer and gets in the way of exercising. Third, the apparatus needed to be repositioned for each exercise. Fourth, there was frequent error in feedback, because we clinicians had no way of standardizing the amount of starting air volume in the blood pressure cuff. Also, the effectiveness of blood pressure cuff depends on the clinician properly placing the air-filled sac to get an accurate reading. Finally, there was no way for the customer to set up a hands-free view of the pressure gauge.

Taking into account these problems with the current device, I developed my own design. I set about making an apparatus that is more efficient, operable by the customer, and easy to put on correctly. When going through the thought process of designing my own apparatus, I realized there is no reason to show the pressure units (so I can use a different manometer), and I realized that constant air volume and a closed system were crucial to the effectiveness of the apparatus. I invented a way to eliminate the bulb for pumping air into the air sac with a separate hose. Also, I needed a belt to go with this so the customer could move between multiple exercises without having to reset the apparatus under the back properly. Since I have patients of all different sizes, I needed an adjustable belt. And since activation of the abdominals can result in expanding the abdomen, I sought a material for the belt with more elasticity than typical nylon belts used in physical therapy in addition to material that can be properly cleaned between uses.

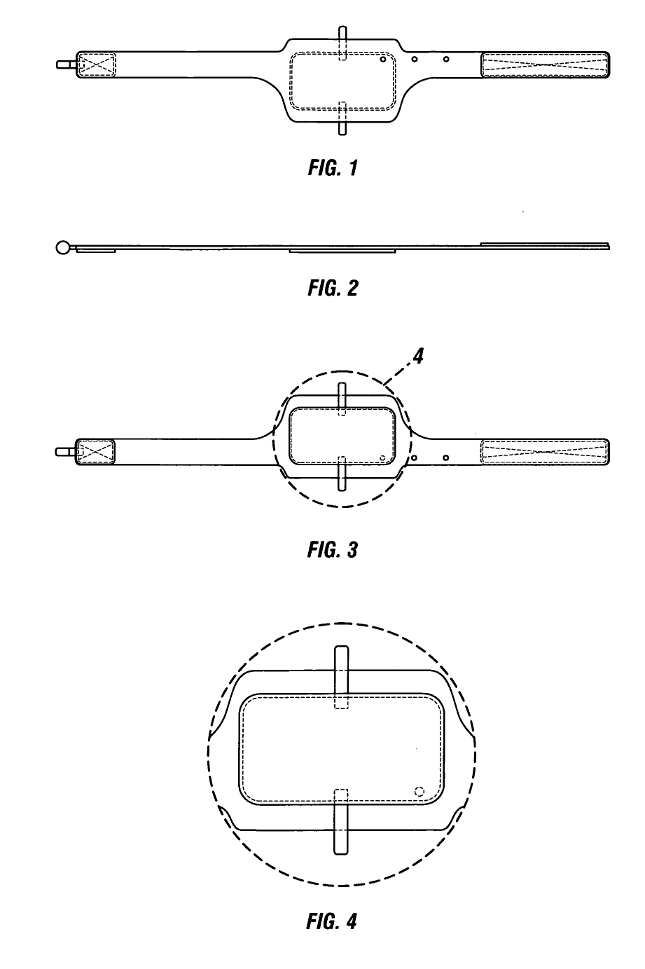

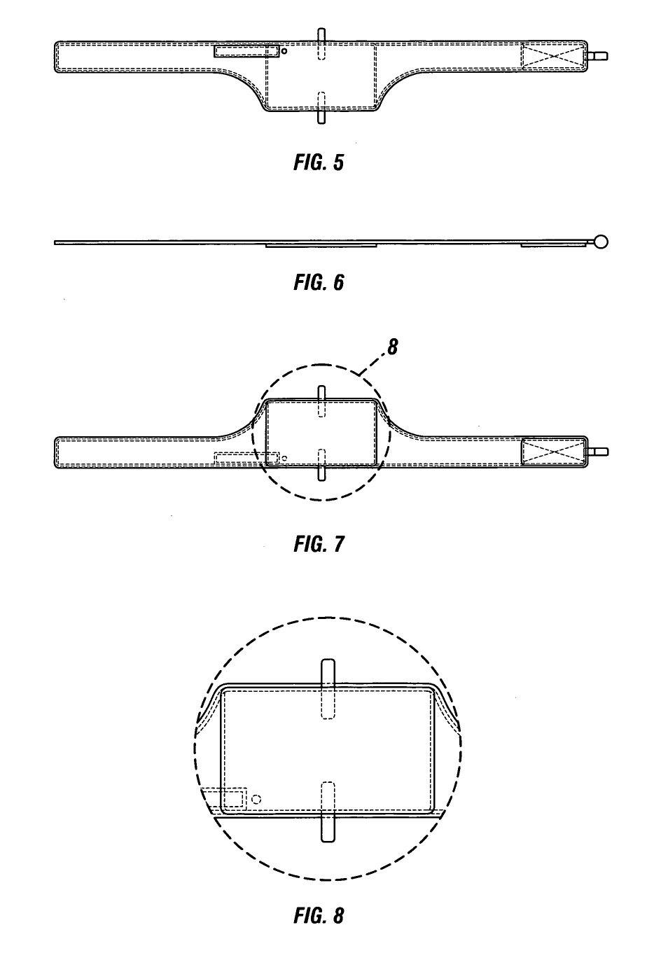

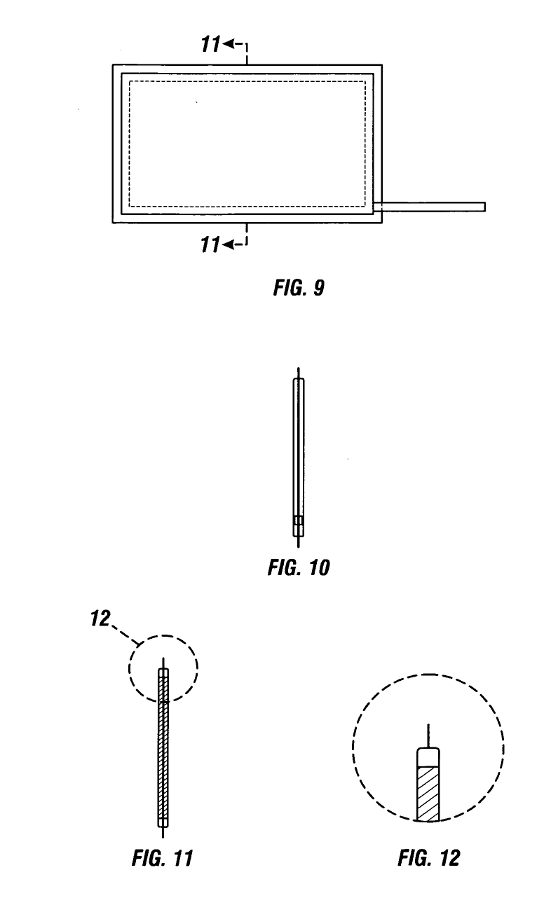

In an exemplary embodiment of the present invention, as shown in FIG. 1, my exercise monitoring apparatus 10 has an expandable waist belt 20, a polyurethane bladder that contains air-filled, compressible foam (not shown) a rubber hose 30, and a manometer 40. This apparatus when placed correctly around the waist and lower back provided an objective biofeedback for the activation of the abdominal and core muscles, throughout various exercises.

The biofeedback was enabled by the use of a manometer 40 attached to the single hose 30 extending from the foam-filled sac that is embedded in a wider section 50 of the waist belt 20. As the transverse abdominals and multifidi were activated appropriately during various exercises, the pressure in the foam filled sac increased and was read on the aneroid dial 60. The pressure increased with correct activation of the abdominals in combination with movement of the upper torso, upper extremity, and/or the lower extremity. There are many advantages in both the design and the utilization of this biofeedback product and concept. The potential advantages are: 1) independence from a clinician to utilize this apparatus correctly which allows for a broader range of people able to utilize this apparatus and concept of training their “core”, 2) chronic back pain decreased, 3) more effective physical therapy for those patients that are trying physical therapy to either put off the need for surgery or as their last resort prior to surgery in many parts of the body (i.e. shoulder, back, knee, hip); 4) the design of the waist belt allowed for consistency of proper placement with each use; 5) the design of the belt with markers indicating proper placement and the long hose allowed persons to be independent from their clinician, strength coach, personal trainer, etc; 6) prevention of acute and chronic injuries (i.e. knee, back, hip, shoulder) due to proper and effective “core” training; and 7) the long hose with a hands-free stand allowed the patient to perform exercises without moving their body in improper ways when attempting to read the dial for feedback during exercises.

FIG. 2 shows more details of the monitoring apparatus 10. As shown, the belt 10 is approximately 42 inches long. In the middle of the belt 10 is the wider section 50 that holds the foam-filled sac 70. To assure proper placement, the sac 70 is shown stitched in place. At either end of the belt 10 are hooks and loop strips of Velcro® 80 and 90. Alternately, the Velcro hooks grasp the Neoprene material alone. Strip 90 is shown as about 12 inches in length and to allows ample adjustment of the belt length. Between strip 90 and the wider portion 50 of the belt 10 is shown a sewn channel 100 in the belt. The rubber hose (not shown) exits the sac 70, travels through the channel 100 and exits the belt (not shown) adjacent the strip 90.

The monitoring apparatus is preferably made from two pieces of Neoprene® that are sewn or glued together. A sac is filled with gas-filled foam and is closed except for an opening to attach the proximal end of a rubber hose. A pocket is partially formed in the middle of the belt and is sewn shut around the sac, except for a small opening for the rubber hose that is then placed in the belt and a channel is sewn around it. A manometer is attached to the distal end of the hose. Nylon tabs are sewn to one end of the belt and to the upper and lower centers of the wider portion of the belt. Lastly, Velcro® hooks are attached to one end of the belt and Velcro loops attached to the other end.

To use the monitoring apparatus, the customer places the belt at the waist with the central tabs substantially aligned with the spine. The customer stretches and overlaps the ends of the belt and presses the Velcro hooks into the Velcro loops to firmly attach the monitoring apparatus. The rubber hose and manometer hang from the side of the belt. The customer positions herself for the exercise and places the manometer where she can see the face of the manometer, optionally clipped on a holder/stand. The customer begins a series of core strengthening exercises and glances at the manometer, which initially indicates improper activation of transverse abdominals. With harder work, the manometer indicates initiation of activation, and finally the manometer indicates proper and consistent activation of transverse abdominals and other core muscles.

### Materials for an Exemplary Embodiment

The hose is made of biocompatible material, including but not limited to rubber.

The manometer is available from a variety of manufacturers, including but not limited to American Diagnostic Corporation. The face of the manometer need not be limited to numbers in terms of mmHg or torr. In fact, these numbers are not very meaningful to most customers and their significance is quickly forgotten. Preferably the face of the manometer is designed to show the preferred pressure range. Other areas of the face can optionally have encouraging words and color-coded ranges.

The material of the belt can be made of any material that is somewhat stretchy but has the strength to hold the foam sac in place. Preferred materials are rubber and synthetic rubber, such as NEOPRENE® polychloroprene.

The belt can be fastened by any of a variety of fasteners, including but not limited to Velcro® hooks and loops, hooks and eyes, buttons and buttonholes, and Velcro® hooks grasping the belt material.

The sac can be made from a flexible, air-proof material such as vinyl or polyurethane. The compressible foam is preferably made of polyethylene open-cell foam. Other materials can be substituted, provided they have the same features, including but not limited to polyurethane foam.

The monitoring apparatus is preferably made from two pieces of Neoprene® that are sewn or glued together. Initially a polyurethane sac is filled with gas-filled polyethylene foam. A pocket is partially formed in the middle wider section of the belt and is sewn shut around the sac, except for a small opening for the rubber hose that is then placed in the belt and a channel is sewn around it. An opening is formed in the side of the belt for the hose to exit and a manometer is attached to the distal end of the hose. Nylon tabs are added to one end of the belt and to the upper and lower centers of the wider portion of the belt. Lastly, Velcro® hooks are attached to one end of the belt and Velcro loops attached to the other end.

To use the monitoring apparatus, the customer places the belt at the waist with the central tabs substantially aligned with the spine. The customer stretches the ends of the belt to overlap and presses the Velcro loops into the Velcro hooks to firmly attach the monitoring apparatus. The rubber hose and manometer hang from the side of the belt. The customer positions herself for the exercise and places the manometer where she can see the face of the manometer, optionally on a holder. The customer begins a series of core strengthening exercises and glances at the manometer, which initially indicates improper activation of transverse abdominals. With harder work, the manometer indicates initiation of activation, and finally the manometer indicates proper and consistent activation of transverse abdominals and core muscles.

### Example 1

A waist belt is made of washable Neoprene® material, in the preferred embodiment, or any elastic type of material that can expand and fit snugly around the body when wrapped around the waist. Waist belt is 42″-55″ long with the capability to fit most adult waist sizes. For fastening the belt, a strip of Velcro® loops is placed along the belt's left end and a strip of Velcro® hooks is placed on the inside of the right end. Vertical markers are placed on the belt to indicate proper placement when belt is wrapped around waist. These can be sewn on the belt (loops) or can be silk screened, painted, or glued on the belt. The waist belt also has a pocket that holds the foam sac made of a same material as the belt.

The hose exits from the pocket through a hole in the exterior surface of the Neoprene® belt on a side. The rubber hose is preferably 39″ long and ¼″ diameter, and it is securely fastened to a manometer to ensure an accurate representation of pressure produced in the auto-inflate air sac during exercise.

The hose exits the pocket and weaves through a tunnel on the Neoprene® belt to ensure manometer and hose are easy to reach.

The pocket is made of similar material as the belt, is sewn on the Neoprene® belt in the middle of the inner side of the belt designed as followed: The pocket is sewn on to the waist belt on all 4 sides to secure the foam sac. There is a 0.25″ hole cut out of the exterior surface of belt inside the top left corner of the pocket to allow the hose to exit the pocket. The single chambered sealed polyurethane pouch has open-celled foam embedded inside with a 1 cm diameter of space between the lateral edges of the foam and the edge of pouch as well as:

The proximal end of the rubber hose inserts into the air sac through top left corner and is sealed to ensure no leakage of air around the hose attachment. The rubber hose is then threaded through a channel between the inside and outside pieces of Neoprene® and out the side of the belt as it sits on the customer's waist. Channeling the rubber hose to the side keeps it out of the way of the exercise. The length of the rubber hose is preferably greater than 30 inches or in a range of about 28 to 45 inches. These lengths were chosen to permit the customer to view the manometer face without substantially turning the face from the midline. Substantial turning impedes proper balanced exercising.

The thickness of the open-celled foam affords a set amount of air volume in air sac at all times. The foam is approximately ¼″-1″ inch thick, with the thickness being adjusted for the resistance of the foam. Foam thickness can be adjusted, depending on its resistance to compression and the effect achieved. As pressure on the air-filled sac increases, the preferred embodiment's manometer attached to the rubber hose displays an increase of pressure.

The manometer's face plate is large for ease of reading by customers of all ages that utilize this product. The diameter is equal to or greater than 2″. The gauge can be any practical shape, such as ovoid, rectangular and other multi-sided structures. The manometer has an indicator needle in front of a multi-color display, such as red, yellow, and green. This is designed to provide biofeedback that customers understand. Red indicates improper activation of transverse abdominals, yellow indicates initiation of activation, and green indicates proper and consistent activation of transverse abdominals and core muscles. These colors can be easily seen so the customer need not substantially turn the head to monitor status.

In another embodiment, the biofeedback is also indicated by a noise that sounds if the pressure is not in the “green area” of the manometer during exercises.

A hands-free metal stand is an optional part of this invention as a separate embodiment on which the manometer is seated. This allows the apparatus to be hands-free and aids the ability to perform the exercises properly.

In another embodiment the hands-free metal stand connects to a large body-size padding for the person to lie on when performing exercises.

In another embodiment the hands-free metal stand is designed as a north-pole magnetic stand to allow the south-pole magnetic manometer to be held on with magnetic forces.

### Clinical Examples

I worked with Client #1, a non-athletic patient who had undergone more than three years of physical therapy for her back pain. She had been taught many times by physical therapists to activate her transverse abdominis with the method of pushing the back into the hand of the therapist while lying supine. But when I repeatedly used the inventive apparatus, she was able to see the “objective” feedback of how much more she needed to activate her abs for the necessary effect. She very quickly became pain free in her lumbar spine during exercise and activities of daily living. In addition, she became able to lift and move more weight and to perform longer cardio exercise. She lost two inches from her waist within first month of use.

Client #2 had been treated by a chiropractor for back pain for almost one year. After using the monitoring apparatus for two weeks of training, she was able to reduce the rate of chiropractic visits. After eight sessions, the individual reported no longer needing any chiropractic visits.

Client #3 was an elite football player who needed to rapidly increase his upper body strength and increase his speed so he could participate in the NFL combine. Using the monitoring apparatus to activate his abdominals correctly, his bench pressing increased 50 lbs and his 40 yd time decreased by 0.03 sec in three weeks, with no other change in his strength and conditioning routine.

Client #4 was a runner with repetitive knee injury causing pain. She learned how to activate her abs with the monitoring apparatus and had a noticeably tighter abdomen and a significant increase pelvic stability and lower body strength after four weeks of use. Knee pain was completely gone within the first two weeks, because her center of gravity was moved backward and the harmful anterior force was removed from knees.

Client #5 was four months post pregnancy, with a loose abdomen. Within 7 visits, she lost most of her baby belly fat and gained abdominal and back muscles she never realized she had

Client #6 was a 63-year-old male who had severe low back pain. Three spinal surgeons told him spinal surgery was his only option. He decided to try the monitoring apparatus as one last opportunity to heal prior to going into surgery. In less than three months of using the monitoring apparatus, his back pain was gone, and surgery was no longer needed. Now with no back pain, he works out intensely and lifts double the amount of weight he had been able to lift in the last five years.

Where the apparatus and procedures described herein for the monitoring apparatus, there may be additional modifications to provide a commercially viable apparatus. As can be seen from the drawing figures and from the description, each embodiment and method of the inventive apparatus and method in accordance with the present invention solves a problem by addressing the need for an improved apparatus to enable athletes and others to properly exercise and build up their abdominal and “core” to avoid injuries and improve strength and physical prowess.

Although specific embodiments have been illustrated and described herein, those of ordinary skill in the art will appreciate that any arrangement calculated to achieve same purposes can be substituted for the specific embodiments or exemplary methods shown. This disclosure is intended to cover any and all adaptations or variations of various embodiments of the invention. It is to be understood that the above description has been made in an illustrative fashion, and not a restrictive one. Combinations of the above embodiments, and other embodiments not specifically described herein will be apparent to those of skill in the art upon reviewing the above description. The scope of various embodiments of the invention includes any other applications in which the above structures and methods are used. Therefore, the scope of various embodiments of the invention should be determined with reference to the appended claims, along with the full range of equivalents to which such claims are entitled.

In the foregoing description, if various features are grouped together in a single embodiment for the purpose of streamlining the disclosure, this method of disclosure is not to be interpreted as reflecting an intention that the claimed embodiments of the invention require more features than are expressly recited in each claim. Rather, as the following claims reflect, inventive subject matter lies in less than all features of a single disclosed embodiment. Thus the following claims, and such other claims as may later be added, are hereby incorporated into the description of the embodiments of the invention, with each claim standing on its own as a separate preferred embodiment.

1. An exercise monitoring apparatus for monitoring pressure generation needed for proper abdominal strengthening, the apparatus comprising
a. A stretchable belt with a first end, second end, a first side, a second side, inside surface, outside surface;
i. the first and second ends having fasteners to attach the belt around the customer's waist, the fasteners being so arranged as to accommodate a variety of waist sizes;
ii. the first side having a small opening; and
iii. the belt having a wider portion in the middle formed by wider surfaces of the fabric, the wider portion forming a pocket with an exit at the small opening;
b. A finable sac containing gas-filled foam, the sac residing in the pocket; and
c. A manometer attached to the sac via a tube that passes out through the small opening, the manometer displaying data on pressure changes as the customer uses the belt.

2. The apparatus of claim 1 wherein the center of the wider portion has a marker to align substantially with an athlete's spine.

3. The apparatus of claim 2 wherein the marker is painted, sewn, silk screened or glued onto the belt surface.

4. The apparatus of claim 1 wherein the belt is formed from Neoprene® polychloroprene or other material with similar characteristics.

5. The apparatus of claim 4 wherein the two surfaces of the belt comprise two at least attached pieces of Neoprene®.

6. The apparatus of claim 1 wherein the tube connecting the foam sac and the manometer is sufficiently long for the person wearing the belt to see the manometer while exercising and without twisting.

7. The apparatus of claim 6 wherein the tube is at least 30 inches long.

8. The apparatus of claim 6 wherein the tube is between 28 and 45 inches long.

9. The apparatus of claim 1 wherein the tube passes from the sac through the belt to a side hole for exiting the belt.

10. The apparatus of claim 1 wherein the manometer has a face with demarcated directions and pressure units.

11. The apparatus of claim 1 wherein the small opening is lengthened to the width of the sac, thereby permitting removal of the sac assembly.

12. The apparatus of claim 1 wherein the fasteners comprise Velcro® hooks and loops, hooks and eyes, buttons, snaps, etc.

13. An exercise monitoring apparatus for monitoring proper abdominal strengthening, the monitoring apparatus comprising
a. A stretchable belt formed from a single piece of Neoprene® with a first end, second end, a first side, a second side, inside surface, outside surface;
i. the first and second ends having fasteners to attach the belt to the athlete's waist, the fasteners being so arranged as to accommodate a variety of waist sizes;
b. A pocket sized to contain a sac filled with compressible foam, the pocket having an inner surface and an outer surface, the inner surface being attached to the outside belt material of the belt, so that the foam sac is compressed against the back in use, the pocket further having an upper open edge; and
c. A manometer attached to the sac via a tube that passes out through the open upper edge of the pocket, the manometer representing data on pressure changes as improper activation, initiation of activation, and proper and consistent activation of transverse abdominals, as the customer uses the belt

14. The apparatus of claim 13 wherein the outer surface of the wider section of the belt has a marker to align with an athlete's spine.

15. The apparatus of claim 14 wherein the marker is painted, silk screened, sewn or glued onto the belt surface.

16. The apparatus of claim 13 wherein the two surfaces of the belt comprise two attached pieces of Neoprene® material.

17. The apparatus of claim 13 wherein the tube connecting the foam sac and the manometer is sufficiently long for the person wearing the belt to see the face of the manometer while exercising and without twisting or having to torque their neck.

18. The apparatus of claim 17 wherein the tube is about 28-45 inches long.

19. The apparatus of claim 13 wherein the fasteners comprise Velcro® hooks and loops, Velcro hooks alone on the fabric, hooks and eyes, buttons, or snaps

20. A method of monitoring and maintaining the proper pressure of the abdominal muscles during exercise, the method comprising
a. providing a apparatus comprising a stretchable, two-layered belt with a wide middle portion for encapsulating a foam sac, the foam sac being attached to a tube whose other end attaches to a manometer, either end of the belt having fasteners, and the middle of the belt having a marker for centering the foam air sac over the spine;
b. putting on the belt by pulling the belt around the midsection;
c. attaching the fasteners for a snug fit;
d. checking to assure that the center marker is over the spine and optionally rearranging the belt until the belt is so positioned;
e. positioning the body of the customer to perform an exercise involving the transversal abdominals or the multifidi; Positioning the manometer so that the customer can see the manometer face without turning the face from the midline; beginning the exercise; and
f. watching the manometer face and adjusting the exercise performance until the manometer face indicates proper and consistent activation of muscles.
